(19) United States
(12) Patent Application Publication
(10) Pub. No.: US 2019/0208917 A1
(43) Pub. Date: Jul. 11, 2019

(54) MEDICAL BED ADAPTED BEDDING AND FURNITURE COVER

(71) Applicant: Ryan Leblanc, Fort Worth, TX (US)

(72) Inventor: Ryan Leblanc, Fort Worth, TX (US)

(21) Appl. No.: 15/984,712

(22) Filed: May 21, 2018

Related U.S. Application Data
(60) Provisional application No. 62/616,378, filed on Jan. 11, 2018.

(51) Int. Cl. A47C 21/02; A61B 5/00

(52) U.S. Cl. CPC A47C 21/02; A47C 31/10; A61B 5/6891

(57) ABSTRACT

The invention described are furniture covers adapted for medical furniture and has particular uses for medical beds. The invention includes headboard covers, and footboard covers, and may also include pillow covers, and mattress covers. Additionally, the furniture covers are adapted to hold wireless sensors for monitoring the patient and recording their vital signs, as well as a call button/light so that the patient may communicate with a medical staff.

### STATEMENT OF PRIORITY

The present invention is related to and claims priority from U.S. Provisional Patent Application No. 62/616,378 entitled: MEDICAL BED ADAPTED FURNITURE COVER by common inventor LeBlanc, filed on Jan. 11, 2018.

### TECHNICAL FIELD

The invention generally relates to medical furniture, and more particularly to bedding and covers for medical furniture that are able to accommodate wireless sensor technology known as the internet of things.

### Problem Statement and History

### Interpretation Considerations

This section describes technical field in detail and discusses problems encountered in the technical field. Therefore, statements in the section are not to be construed as prior art.

### Discussion of History of the Problem

Medical beds (also called hospital beds, therapeutic beds, home care beds, and similar terms known to those in the medical profession, and such existing devices, dimensions, and descriptions are hereby incorporated by reference herein) have evolved since the 1860s, and, from hospitals, to elder-care facilities, to homes, are used in many types of settings. Originally, medical beds were basically beds designed to prevent patients from being hurt by non-medical bedding, to provide a sturdy platform for medical professionals to assist patients, and to be easily cleaned. Today, medical beds support the repeated pressures of CPR being administered on a bed, adjust to place a patient at an appropriate incline, and support a vast variety of medical apparatuses such as monitors, intravenous bags, and the like.

Currently, clips are used to hold medical accessories (such as call lights or buttons) to hospital beds, and sensors are frequently tapped directly to a patient's skin, and their wires are draped over various structures adapted for medical beds. However, there is not currently an effective method to secure sensors and devices to a patient (or make them accessible to the patient) without disturbing the patient by waking them up or, when removing the sensors, ripping their skin. Clips are sometimes used. However, clips can fall off a patient. And, when monitoring clips fall off a patient the monitoring is cut-off, a false emergency is triggered to which the medical staff must respond. Additionally, clips can snag onto surrounding fabric, tearing the fabric, and the wires that connect the sensors to the monitors tangle around the patient, and can cause blood-flow to be cut-off, and other injuries to the patient. Furthermore, clips sometimes break, and medical accessories (such as call lights or buttons), which are attached or tied to the bed can cause a tripping or choking hazard.

An additional issue with medical beds is that they psychologically impact a patient. Stress induced from a hospital stay, or from the disruption of one's home, can impact the health and recovery of the patient negatively. Accordingly, there is the need for medical bedding systems that look-and-feel more like home.

Accordingly, the invention consists of adapted medical furniture bedding that facilitates sensor use, and reduces patient stress.

### SUMMARY OF THE INVENTION

In one aspect, the invention is an apparatus for covering a headboard of a medical bed. In this aspect the invention includes a fabric-like slipcover sized to fit over a headboard of a medical bed, where the slipcover has at least a first wireless sensor pocket positioned on the slipcover such that when the slipcover is on the headboard of the medical bed, the first wireless sensor pocket is positioned just above a top surface of a mattress on/in the medical bed's bedframe. The first wireless sensor pocket is adapted to hold a first medial monitoring device, such as a wireless sensor. Additional advantages may be found by incorporating wireless sensors within the threading of the slipcover itself, such as are achievable by printing a circuit on fabric. Preferably, the slipcover also includes a first call-light/call-button loop positioned along a side-seam of the slipcover, which is adapted to accept a patient call-light/call-button.

In another aspect, the invention is an apparatus for covering a footboard of a medical bed. In this aspect the invention includes a fabric-like slipcover sized to fit over a footboard of a medical bed, where the slipcover has at least a first wireless sensor pocket positioned on the slipcover such that when the slipcover is on the footboard of the medical bed, the first wireless sensor pocket is positioned just above a top surface of a mattress on/in the medical bed's bedframe. The first wireless sensor pocket is adapted to hold a first medial monitoring device, such as a wireless sensor. Additional advantages may be found by incorporating wireless sensors within the threading of the slipcover itself, such as are achievable by printing a circuit on fabric. Alternatively, the invention may include a padding lining the interior of the footboard slipcover to provide patient comfort, and to facilitate data reading by a wireless monitoring device in the first wireless sensor pocket of the slipcover.

### BRIEF DESCRIPTION OF THE DRAWINGS

Various aspects of the invention and its embodiment are better understood by referring to the following detailed description. To understand the invention, the detailed description should be read in conjunction with the drawings, in which:

FIG. 1 illustrates an exploded view of the inventive medical bed;

FIG. 2 illustrates one embodiment of a sensor pocket;

FIG. 3 illustrates one exemplary wireless sensor;

FIG. 4 illustrates an inventive pillow cover;

FIG. 5 illustrates an inventive bed cover;

FIG. 6 illustrates an inventive headboard slipcover;

FIG. 7 illustrates an inventive footboard slipcover; and

FIG. 8 illustrates an alternative embodiment of the inventive medical bed.

### DESCRIPTION OF AN EXEMPLARY PREFERRED EMBODIMENT

### Interpretation Considerations

While reading this section (An Exemplary Embodiment, which describes the exemplary embodiment of the best mode of the invention, hereinafter referred to as “exemplary embodiment”), one should consider the exemplary embodiment as the best mode for practicing the invention during filing of the patent in accordance with the inventor's belief. As a person with ordinary skills in the art may recognize substantially equivalent structures or substantially equivalent acts to achieve the same results in the same manner, or in a dissimilar manner, the exemplary embodiment should not be interpreted as limiting the invention to one embodiment.

The discussion of a species (or a specific item) invokes the genus (the class of items) to which the species belongs as well as related species in this genus. Similarly, the recitation of a genus invokes the species known in the art. Furthermore, as technology develops, numerous additional alternatives to achieve an aspect of the invention may arise. Such advances are incorporated within their respective genus and should be recognized as being functionally equivalent or structurally equivalent to the aspect shown or described.

A function or an act should be interpreted as incorporating all modes of performing the function or act, unless otherwise explicitly stated. For instance, sheet drying may be performed through dry or wet heat application, or by using microwaves. Therefore, the use of the word “paper drying” invokes “dry heating” or “wet heating” and all other modes of this word and similar words such as “pressure heating”.

Unless explicitly stated otherwise, conjunctive words (such as “or”, “and”, “including”, or “comprising”) should be interpreted in the inclusive and not the exclusive sense.

As will be understood by those of the ordinary skill in the art, various structures and devices are depicted in the block diagram to not obscure the invention. In the following discussion, acts with similar names are performed in similar manners, unless otherwise stated.

The foregoing discussions and definitions are provided for clarification purposes and are not limiting. Words and phrases are to be accorded their ordinary, plain meaning, unless indicated otherwise.

### Description of the Drawings, A Preferred Embodiment

The invention includes systems and devices for adapting medical beds and other medical furniture to better accommodate patients and provide improved patient care. Generally, the invention is a system that comprises fabric or fabric-like covers for medical furniture that include pockets or pouches that are adapted to secure wireless sensors and medical monitoring equipment therein, and which may be customized to have an appearance that is pleasing to a patient. In particular, the invention has particular applicability to medical beds of all types such as hospital beds, therapeutic beds, home care beds, nursing home beds, assisted care living beds and medical beds for home use, but of course the teachings can be applied to coverings for a variety of other medical furniture.

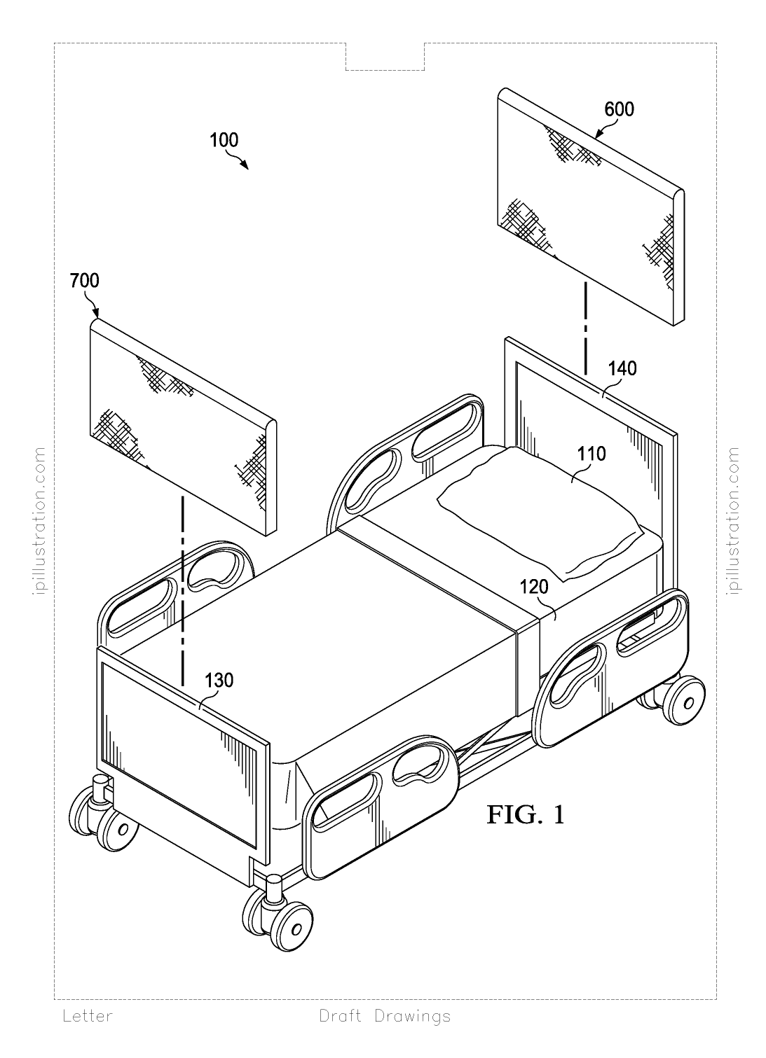

FIG. 1 illustrates an exploded view of a preferred embodiment of an inventive medical bed system 100. The augmented medical bed system includes a medical bed having a footboard 130, a headboard 140, a pillowcase 110 for covering a pillow, a mattress cover 120 such as a fitted sheet, a headboard cover 600 and a footboard cover 700. Preferably, each of the fabric-like structures of the pillowcase 110, mattress cover 120, headboard cover 600 or footboard cover (collectively “furniture covers”), is adapted to accommodate a wireless sensor, such as with a sensor pocket or pouch. Fabric-like structures are made of fabric-like materials, such as fleece, cotton, flannel, microfiber, nylon, polyester, silk, denim, leather, quilt, suede, micro-suede, spandex, corduroy, flax, wool, ramie, down, fur, neoprene, polymers, vinyl, recycled cotton, recycled paper, burlap, and fibers of natural, cellulose, or synthetic form.

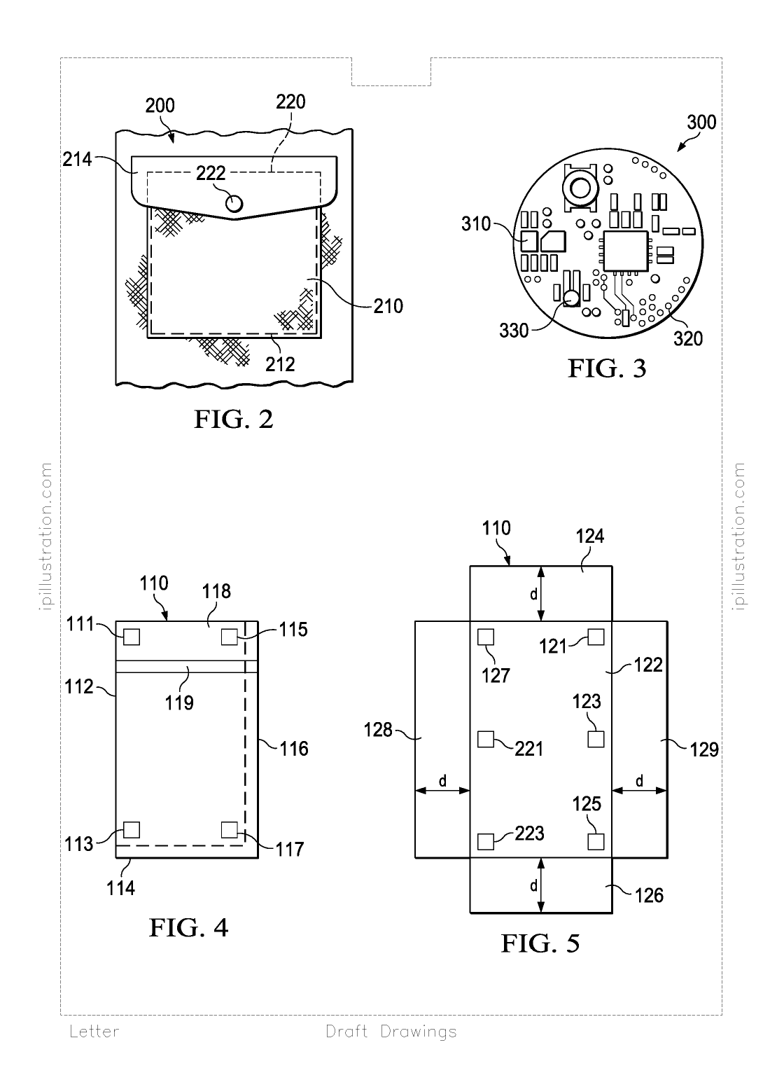

Accordingly, FIG. 2 illustrates one embodiment of a wireless sensor pocket 200. The wireless sensor pocket 200 shown in FIG. 2 comprises a fabric piece 210 sewed into a furniture-cover 230 made of fabric-like material via a seam 212 to create an opening 220. The opening 220 is closed via a flap 214 and is secured by a snap 222. Of course, although the wireless sensor pocket 200 shown in FIG. 2 is created by sewing the fabric piece 210 to the furniture cover 230, other fabric-attachments are possible, such as adhesives, and heat-activated fabrics, for example. Additionally, although the snap 22.2 is shown, other attachment such as magnets, clips, buttons, snaps, tape, hook and loop (Velcro®), burrs, touch fasteners, laces and/or ties may also be used.

FIG. 3 illustrates one exemplary wireless sensor 300. The wireless sensor 300 includes a sensor 310 which detects one or more data types, such as temperature, weight, a pulse rate, bio-hazards (such as strep, staph, or other infectious agents), and a host of other environmental characteristics. The wireless sensor 300 also includes a wireless communication hardware, such as a Bluetooth low energy transceiver 320, and a power source such as a watch-sized battery. Preferably, a user-feedback means, such as a Light Emitting Diode (LED) 330 is provided in the wireless sensor to provide information to a user or operator (such as a nurse) to indicate that the device is working, and/or to indicate that a particular physical property (including chemical and biological agents) has been detected. One exemplary wireless sensor is the MbientLab's Metawear® platform. The wireless sensor 300 preferably communicates with a wireless monitor, which is able to store data as well as alert staff in the event that predetermined data and/or data patterns are measured.

FIG. 4 illustrates an inventive pillow cover 110 (also called a pillowcase, and other words known in their respective regional dialects). The pillow cover 110 is made of a fabric-like material and includes edges defined by a fold 112 of the material which terminates in a first raw edge 114 adjacent to the fold 112, and a second raw edge 116 opposite the fold 112. The raw edges 114, 116 are sealed with a sewn seam illustrated by the dotted lines. Pillows typically terminate in an opening, or, as illustrated here, in a cuff 118. The cuff 118 is made from a length of the fabric-like material opposite of the first raw edge 114, the cuff 118 being folded over the opening in the pillow cover 110. Preferably, a band 119 of fabric-like material is folded from the cuff 118 itself, and sewn to the cuff 118. Also illustrated in FIG. 4 are a first wireless sensor pocket 111, second wireless sensor pocket 113, third wireless sensor pocket 115 and fourth wireless sensor pocket 115 which are each preferably sewn into each of the four corners of the pillow cover 110 as shown. Although the wireless sensor pockets 111, 113, 115 and 117 are shown on the exterior of one side the pillow cover 110, it is understood that the wireless sensor pockets 111, 113, 115 and 117 may be affixed to the interior of the pillow cover. Additionally, the pillow cover 110 may have wireless sensors 111, 113, 115 and 117 on the side of the pillow cover 110 that is not illustrated in FIG. 4, in which case the pillow cover 110 would have eight total wireless sensors.

FIG. 5 illustrates an inventive mattress cover 120, here presented as a fitted sheet. The mattress cover 120 comprises a rectangular fabric-like material, with a generally rectangular mattress portion 122 with dimensions of (or slightly larger than) the mattress to be covered. Additionally, the mattress cover 120 extends approximately the depth (“d”) of the mattress to be covered beyond the mattress portion 122 in each of the rectangular dimensions, as illustrated by the dashed lines in FIG. 5. Typically a square portion of each corner of the mattress cover 120 is removed from the fabric-like material to create a first flap 124, a second flap 126, a third flap 128, and a fourth flap 129. Although beyond the scope of the present discussion, in practice, each edge of each flap 124, 126, 128 and 129 are sewn or otherwise affixed to each other to form a seam at each corner of the mattress cover 120.

Also seen in FIG. 5 are a first wireless sensor pocket 121, second wireless sensor pocket 123, third wireless sensor pocket 125 along the first side of the mattress cover 120, as well as a fourth wireless sensor pocket 127, a fifth wireless sensor pocket 221, and a sixth wireless sensor pocket 223 along the second side of the mattress cover 120. Preferably, one of four wireless sensor pockets is sewn into each of the four corners of the mattress cover 120, and one wireless sensor pocket is sewn approximately half-way down each side, as shown.

Although the wireless sensor pockets 121, 123, 125, 127, 221 and 223 are shown on the top side the mattress cover 120, it is understood that the wireless sensor pockets may be affixed underneath the mattress cover 120 as well. Of course, the mattress cover may have additional wireless sensor pockets in different locations as needed to monitor a patient.

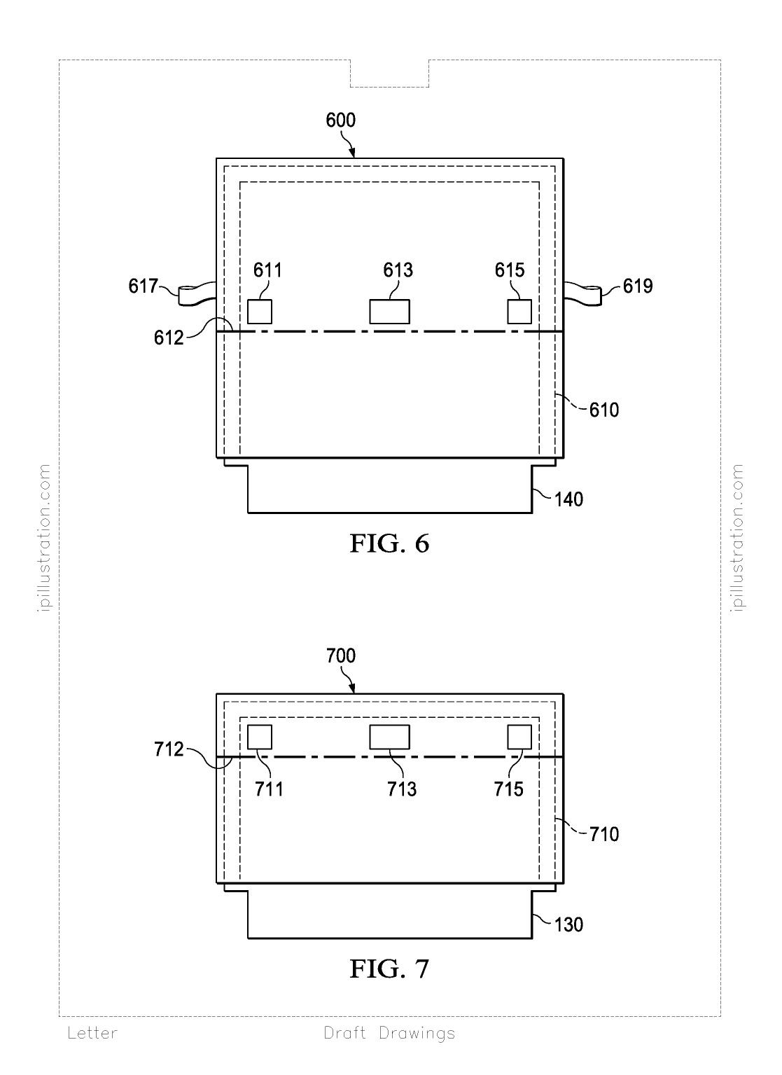

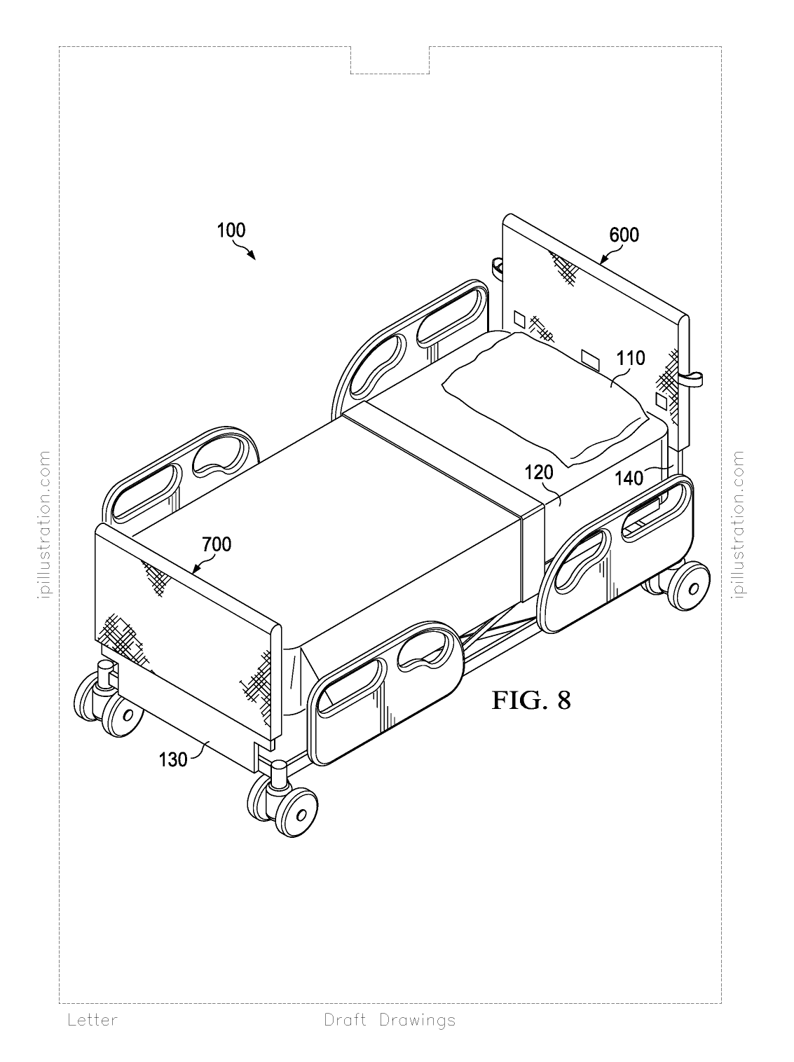

Now simultaneous reference is made to FIGS. 6 and 7 in which FIG. 6 illustrates an inventive headboard cover (slipcover) 600, and FIG. 7 illustrates an inventive footboard cover (slipcover) 700. Each of the headboard slipcover 600 and the footboard slipcover are preferably made of fabric-like materials 705 that are constructed to fit their respective headboard 140 and footboard 130 (the dotted lines 610 of FIG. 6 and 710 of FIG. 7 show the location of the structure of the headboard 140 and footboard 130, respectively). By contrast, the dashed lines (“mattress lines”) 612 of FIG. 6 and dashed lines (“mattress lines”) 712 of FIG. 7 illustrate the approximate location of the top of a mattress of a medical bed. The headboard slipcover 600 includes a plurality of wireless sensor pockets 611, 613 and 615 positioned along the mattress line 612, and, preferably, the wireless sensor pocket 613 is large enough to also hold medial monitoring and signaling devices, such as at least a call light and/or call button (not shown but readily understood by those of ordinary skill in the medical care arts). In an embodiment patients may easily attach, or detach and release a call light or call button from the headboard slipcover 600.

The headboard slipcover 600 also includes a first call-light/call-button loop 617 and a second call-light/call-button loop 619, which may either (or both) accept a patient call-light/call-button which is used by a patient to request the attention of care staff or medical staff. Each of the first and second call-light/call-button loops 617, 619 are preferably made of the same or similar material as the headboard slipcover 600, and are attached to the headboard slipcover via sewing, or adhesive, for example. Of course, other means of attaching a call-light/call-button are within the scope and meaning of the invention, such as embodying the call-light/call-button loops 617, 618 as fabric or metal eye-loops or grommets that are attached directly to an ear (not shown) that protrudes from the headboard slipcover 600.

Similarly, the footboard slipcover 700 includes a plurality of wireless sensor pockets 711, 713 and 715 positioned along the mattress line 712, and, preferably, the wireless sensor pocket 713 is large enough to also hold medial monitoring and signaling devices. Further, although not illustrated, it may be desirable to provide padding within the footboard slipcover 700 to provide patient comfort, and to allow any wireless monitoring devices of the footboard slipcover 700 to indirectly contact the patient's mattress, which facilitates the monitoring of some physical and biological factors.

Of course, although slipcovers are discussed and disclosed, the invention is no limited to slipcover structures when covering a headboard or footboard. For example, one may utilize an attachment, such as buttons, snaps, or a Velcro®-attached single sheet, for example, to attach a headboard cover or footboard cover to the headboard, or footboard, respectively.

FIG. 8 illustrates the inventive medical bed 100 of FIG. 1, this time with the headboard cover 600 and footboard cover 700 placed on the headboard 140 and footboard 130, respectively. From FIG. 8 it is seen that the headboard cover 600 has a first design 690 thereon, and that the footboard cover 700 also has a second design thereon 790 (the first design 690 and the second design 790 are collectively referred to as “designs”).

Designs include customized designs selected by or for a patient, including pictures, photos, logos, words, patterns, for example, and may be created on the headboard cover 600 or footboard cover 700 via embroidery, decals, stickers, iron-on transfers, patches, heat transfer imprinting, and other transfers to fabrics. Additionally, upon reading this disclosure modifications of and specific images for the covers 600, 700 will become readily apparent to those of skill in the fabric, screen-printing, and other arts without departing from the teaching of the invention.

Although the invention has been described and illustrated with specific illustrative embodiments, it is not intended that the invention be limited to those illustrative embodiments. Those skilled in the art will recognize that variations and modifications can be made without departing from the spirit of the invention. Therefore, it is intended to include within the invention, all such variations and departures that fall within the scope of the appended claims and equivalents thereof.

I claim:

1. An apparatus for covering the headboard or footboard of a medical bed, comprising:
a fabric-like slipcover sized to fit over a headboard of a medical bed;
at least a first wireless sensor pocket positioned on the slipcover such that when the slipcover is on the headboard of the medical bed, the first wireless sensor pocket is just above a top surface of a mattress;
the first wireless sensor pocket is adapted to hold a first medial monitoring device;
the slipcover also comprising a first call-light/call-button loop adapted to accept a patient call-light/call-button, the first call-light/call-button loop being positioned along a side-seam of the slipcover.

2. An apparatus for covering the headboard or footboard of a medical bed, comprising:
a fabric-like slipcover sized to fit over a footboard of a medical bed;
at least a first wireless sensor pocket positioned on the slipcover such that when the slipcover is on the footboard of the medical bed, the first wireless sensor pocket is just above a top surface of a mattress; and
the first wireless sensor pocket is adapted to hold a first medial monitoring device.

3. The apparatus of claim 2 further comprising a padding lining the interior of the footboard slipcover to provide patient comfort, and to facilitate data reading by a wireless monitoring device in the first wireless sensor pocket of the slipcover.
